(19) United States
(12) Patent Application Publication
(10) Pub. No.: US 2025/0082914 A1
(43) Pub. Date: Mar. 13, 2025

(54) SWALLOWABLE, SELF-EXPANDABLE DRUG DELIVERY DEVICE AND USES THEREOF

(71) Applicant: ETH ZURICH, ZURICH (CH)

(72) Inventors: JEAN-CHRISTOPHE LEROUX, ZURICH (CH); ZHI LUO, SHENZHEN, GUANGDONG (CN)

(21) Appl. No.: 18/725,377

(22) PCT Filed: Jan. 4, 2023
(86) PCT No.: PCT/IB2023/050070

§ 371 Date: Jun. 28, 2024

(30) Foreign Application Priority Data
Jan. 6, 2022 (EP) 22150416.0

(51) Int. Cl. A61M 31/00; A61K 9/00; A61K 9/48

(52) U.S. Cl. CPC A61M 31/002; A61K 9/0053; A61K 9/4808

(57) ABSTRACT

A swallowable, foldable and self-expandable oral drug delivery tubular device, the device: comprising an outer peripheral surface; having an expanded length of about 2 cm to about 6 cm; having an expanded diameter of about 0.45 cm to about 6 cm; and comprising at least one cavity enclosing a drug, the at least one cavity facing outward from the outer peripheral surface of the device.

### CROSS REFERENCE TO RELATED APPLICATIONS

This application is a PCT application Serial No PCT/CA2023/05* filed on Jan. 4, 2023 and published in English under PCT Article 21 (2), which itself claims benefit of European patent application Serial No. 22150416.0, filed on Jan. 6, 2022. All documents above are incorporated herein in their entirety by reference.

### FIELD OF THE DISCLOSURE

The present disclosure relates to a swallowable and self-expandable drug delivery device. More specifically, the present disclosure is concerned with a swallowable and expandable drug delivery tubular device and uses thereof in non-parenteral drug delivery.

### BACKGROUND OF THE DISCLOSURE

Improving the oral absorption of poorly bioavailable drugs, especially of macromolecules such as peptides, proteins and nucleic acids, is a long-standing goal in pharmaceutical sciences. A major challenge is that the gastrointestinal (GI) tract presents various physiological barriers, which hinder the absorption of drug molecules.

Great efforts have therefore been devoted to developing novel non-parenteral drug delivery systems for these macromolecular drugs (M. Sam, D. Brayden. “Formulation strategies to improve the efficacy of intestinal permeation enhancers.” Adv. Drug Deliv. Rev (2021): 113925). Success has been scarce with only a few oral formulations for systemic delivery of peptides having entered clinical trials over the past 30 years, resulting in merely a handful reaching the market. The oral bioavailability of most of these drugs remains very low (generally <1%).

More particularly, various chemical excipients have been explored to protect the macromolecule drugs from degradation and enhance their absorption by transiently disrupting the barrier function of the GI epithelium. Two permeation enhancers, salcaprozate sodium (SNAC) and sodium caprate (C10), are the most investigated examples. However, the relatively low potency, rapid dilution and absorption have made it difficult to maintain high concentrations of these permeation enhancers at the intestinal mucosa. Therefore, typically a high dose of permeation enhancers is given (e.g., 300 mg SNAC to deliver 3 mg of semaglutide) to achieve a low oral bioavailability in the range of 1%. While protease inhibitors (to reduce premature degradation) such as citric acid have been generally recognized as safe, an important drawback of other inhibitors is the impaired digestion of dietary proteins and other possible side effects such as systemic intoxication and pancreas hypertrophy (Krauland et al. 2004; Melmed et al., 1976).

Various techniques exploiting the physical forces to overcome the mucosal barriers have also been tested. These devices rely on magnetic, mechanical, electrochemical, and acoustic forces to enhance absorption. Currently most of these methods are still in the proof-of-concept and pre-clinical stages. There are several challenges that need to be addressed before their clinical translation, such as safety, efficacy, robustness, and costs. Several attempts have utilized microneedle-equipped devices for the delivery of macromolecules in the GI tract. For example, dosage forms composed of an actuation device equipped with microneedles that directly puncture the GI tract mucosa and deliver macromolecular drugs. The complexity of the system, robustness and long-term safety of this technology requires more in-depth investigation before their successful translation. Such systems are also associated with a high variability as the injections can be unsuccessful (Dhalla et al., 2021). Microfabricated devices with dispersible micrometer sized drug reservoirs have been developed. The idea is that an increased surface area enables higher contact with the intestine allowing localized release of drugs and excipients. However, so far, such technology has not demonstrated satisfying results in vivo, likely due to the lack of retention and proper orientation of the microcontainers in the mucus layer. The microcontainers have also been integrated onto an elastic foil structure to address the orientation issue. However, the size of the expanded states could not be controlled, and the bioavailability of delivered protein and peptide drugs are below 1% even together with permeation enhancers and protease inhibitors. Another strategy based on superporous hydrogels was proposed to achieve site specific and local release of drugs in the GI tract. The large swelling ratio of these hydrogels enabled the mechanical transient fixation of the drug formulation onto the intestinal wall. A series of in vitro and ex vivo experiments were performed to correlate the mechanical pressure generated by the hydrogel to the paracellular permeability of different drugs. However, the absorption enhancement effect was not very high (Dorkoosh et al., 2002). It is also unclear how the food contents affect the performance of these swellable formulations. There are several other approaches using physical forces to deliver macromolecular (e.g., peptide/protein) drugs, such as ultrasonication, and iontophoresis. However, currently these technologies could not be integrated into a stand-alone swallowable capsule and rely on external devices, which would likely reduce patient compliance and increase the costs.

As a result, novel and more efficient non-parenteral drug delivery strategies are of great interest to the pharmaceutical industry.

There is a need for non-invasive (e.g., needle free) non-parenteral drug administration systems for enhancing systemic delivery, particularly of macromolecular drugs.

The present description refers to a number of documents, the content of which is herein incorporated by reference in their entirety.

### SUMMARY OF THE DISCLOSURE

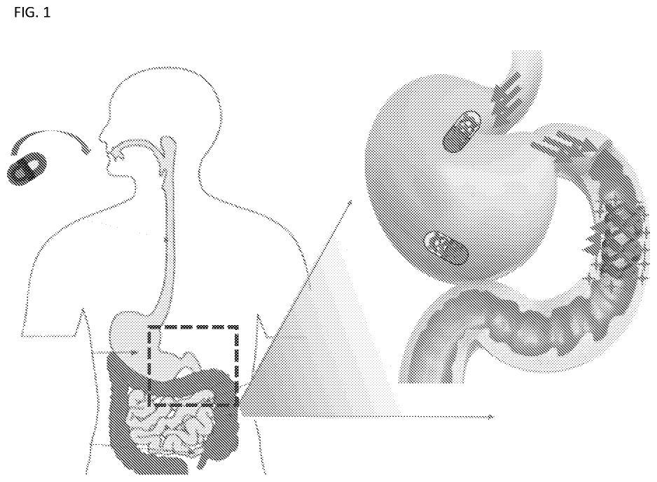

The present disclosure presents a swallowable device designed to release drugs in close proximity to the intestinal wall (or touching it). This novel device comprises a foldable and expandable, optionally degradable tubular structure, with a drug casted into compartments located along external surfaces of the device. The device is folded into an enteric capsule, which selectively releases the device in the GI tract (e.g., small intestine). There, the device expands both radially and axially into its original shape, and exposing the drug to the intestinal epithelium/mucosa (FIG. 1). The physical interaction allows the targeted drug release and the establishment of a sharp local drug concentration gradient, which facilitates its diffusion through the intestinal absorptive barrier.

### Tubular Device

### Tubular Device Shape

### General Tubular Device Shape

The device of the present disclosure has a tubular structure comprising at least one drug-containing cavity (i.e., one (a single) or a plurality/array of at least 2, at least 3, at least 4, at least 5, at least 6, at least 7, at least 8, at least 9 or more cavity(ies)) located on or in (an) outer peripheral surface(s) of the tubular structure. The at least one cavity being located on or in an outer peripheral surface of the device so that, when after the device is orally administered to the subject, and the device is coaxially positioned in the GI tract (e.g., in the intestine), the at least one cavity's opening faces the small intestine epithelium and touches it or is in close proximity thereto. When the device comprises more than one cavity, cavities can be spaced over the device's outer peripheral surface.

Devices of the present disclosure can comprise a simple foldable tubular (or hollow polygonal prism) mesh, or a generally tubular structure comprising a central tube (meshed or solid) or rod from which foldable guiding members (e.g., wings, hollow elliptic or round cylinders or hollow prisms) radially extend outward, along the length of the tube or rod. The device's diameter and its length are adapted to guide/position the device coaxially in the GI tract, and the at least one cavity is located on the device's outer peripheral surface.

The term “tubular structure” or “generally tubular structure” is meant to convey herein that the device is generally tubular but is not necessarily a cylinder per se. Without being so limited, the device may take the form of a cylinder/tube or a hollow polygonal prism comprising at least 4 faces (or 5, 6, 7, 8, 9 or more faces) along the device's length, the generally cylindrical or polygonal prism shape of the device being formed either by (1) a single foldable tubular or hollow polygonal prism; or by (2) the combination of the foldable guiding members radially extending from a central tube/rod. It is also meant to convey that the outer peripheral surface, if the device is observed from a top view (or bottom view) may form a circle or a polygon comprising at least 4 sides (or 5, 6, 7, 8, 9 or more sides). For example, if the device comprises 5 foldable guiding members, and if the outer peripheral surfaces of each of the foldable guiding members is flat (instead of being convex), then the device forms a pentagonal prism. This pentagonal prism is generally hollow although it comprises (i) partitions formed by the sides (i.e., ten sides) of the foldable guiding members radially extending outward from the central tube or rod, and (ii) a central tube or rod. Within the present disclosure, such shape is referred to as a tubular structure.

In embodiments where the device consists of a simple foldable tube (or hollow polygonal prism), the device's outer peripheral surface is the tube's external wall.

In embodiments where the device comprises foldable guiding members (e.g., wings, hollow elliptic or round cylinders or hollow prisms) radially extending from a central tube or rod, each foldable guiding member (each being independently meshed or solid) comprises an outer peripheral surface. The combined outer peripheral surfaces of each foldable guiding member of the device form the device's outer peripheral surface.

In specific embodiments, foldable guiding members radially extending outward from a tube consist of wings radially extending outward from a central tube/rod, the free end of each wing being shaped so as to form a surface facing the GI epithelium (an outer peripheral surface). More particularly, the shape of the wing's free end can be folded so as to form a surface facing the GI epithelium (an outer peripheral surface), or form a T-shape, the horizontal bar of which forms a surface facing the GI epithelium (an outer peripheral surface). In specific embodiments, foldable guiding members radially extending from a central tube/rod consist of hollow elliptic or round cylinders or hollow prisms (e.g., trapezoidal prisms). In embodiments where the foldable guiding members are hollow prisms, two sides (e.g., non-parallel) of each prism radially extend outward from the central tube/rod, the central tube/rod surface between these two sides forming a third side of the trapezoidal prism, and a fourth side forming an outer peripheral surface. The outer peripheral surface of each prism can be flat or convex (or be subdivided into multiple sides forming a generally convex shape) so as to touch or be in close proximity with the GI tract epithelium. In specific embodiments, the plurality of guiding members radially extending outward from a tube/or are different and may comprise a combination of wings with free end folded and/or T-shaped, hollow elliptic cylinders, hollow round cylinders and/or hollow prisms. In embodiments of the device comprising foldable guiding members (e.g., wings, hollow cylinders or prisms) radially extending from a central tube or rod, outer peripheral surfaces of foldable guiding members contain drug cavities.

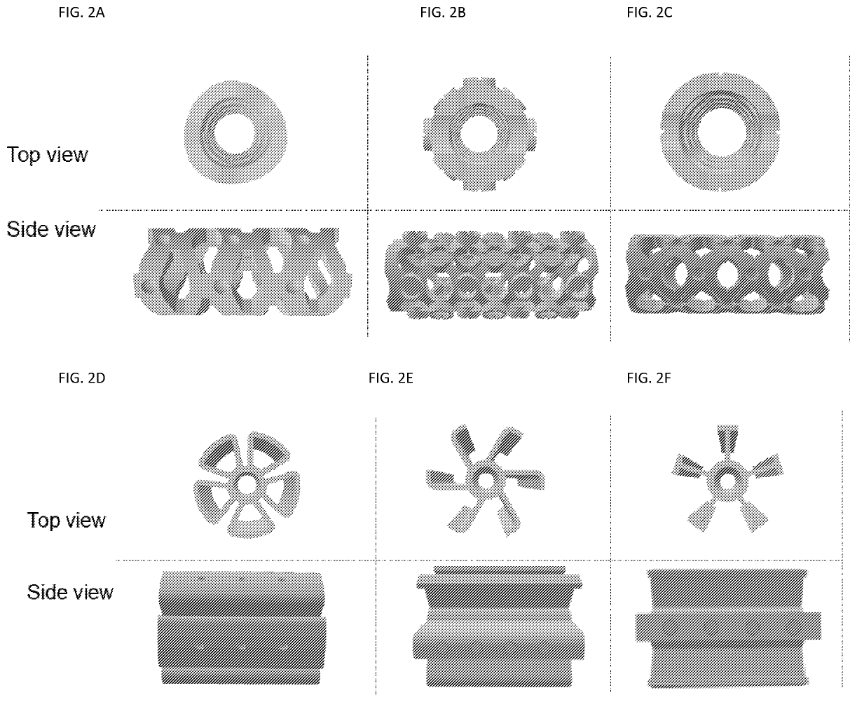

Exemplary devices of the present disclosure are shown comprising a tubular mesh (See FIGS. 2A-C for example), or a tubular structure comprising a central tube or rod (e.g., meshed or not) from which foldable guiding members radially extend (e.g., wings) (see FIGS. 2E-2F for example) and hollow prisms (see hollow trapezoidal prisms in FIG. 2D for example).

The thickness of the device material is designed to enable its easy folding. Further, its thickness is small enough to allow food and fluid transit. In a specific embodiment, the device material has a thickness of about 0.5 mm to about 4 mm.

### Drug Cavities and Holes

The at least one drug cavity of the device has a volume and is in fluid communication with at least one opening (e.g., cup-shaped) to the outside. In devices containing a plurality of cavities, the term “total cavity volume” refers to the combined volumes of all the device cavities. The total cavity volume is of a size sufficient to contain the drug formulation (and any used excipient) intended for delivery.

At least one, and up to all cavities in the device contain(s) a drug formulation. If the device contains two or more cavities that contain a drug formulation, the drug formulation may be identical or different in the two or more cavities. For example, a fraction of the one or more cavities may comprise a first drug formulation, and other cavities may comprise another drug formulation.

The drug cavity(s) is (are) designed so as to accommodate the desired amount of drug and excipient(s) and/or carrier(s). Each drug has specific delivery requirements (e.g., faster or slower release). The present disclosure encompasses the use of cavity sizes, shapes and locations to tailor different drug deliveries.

As indicated above, drug cavities can be located on various parts of the outer peripheral surface of the device. Without being so limited, in specific embodiments, they can be spaced around the device's outer peripheral surface (e.g., to provide 360-degree release of the drug to the intestinal mucosa) and/or along its length, including on the outer peripheral surfaces of one or more of the foldable guiding members.

The drug cavities can optionally be spaced by holes in the outer peripheral surface (meshed outer peripheral surface). Drug cavities and holes can be distributed in any sequence on the device's outer peripheral surface (e.g., a sequence/line of 1 cavity, followed by 1 hole; 1 cavity, 2 holes; 2 cavities, 1 hole; 2 cavities, 2 holes; etc.; a drug cavity surrounded by 4 holes, etc.). The drug cavities can have any shape. Without being so limited, they can be cup-shaped (e.g., FIGS. 2A-D and F), hemispherical, ellipsoid, cubic, cuboid, conic or pyramidal (the point constituting the bottom of the cavity), cylindric/tubular (FIG. 2E), polyhedrons (5, 6, 7, 8, 9, 10 sides, or more), shaped as channels (e.g., FIG. 2C), or irregularly shaped. The concave portion of the cavities can be located completely or partially below the device's outer peripheral surface, with openings on or above the device's outer peripheral surface (see e.g., FIGS. 2A-D and F). The concave portion of the cavities can alternatively be partially or completely above the device's outer peripheral surface (see e.g., FIG. 2B). In specific embodiments, devices of the present disclosure can be meshed. The holes forming the mesh structure can have any shape. Without being so limited, the holes can be round (e.g., FIG. 2B), semi-circular, oval or diamond-shaped (e.g., FIGS. 2A and 2C), square, rectangular, trapezoid, triangular, polygonal (with 5, 6, 7, 8, 9, 10 sides, or more), shaped as channels (e.g., FIG. 2C), or irregularly shaped. Each device can comprise a combination of different cavities and, eventually, holes.

The foregoing features are referred to herein as the device shape.

The device shape largely contributes to the device's proper positioning in the intestine and the proper delivery of the drug through the GI tract epithelium.

### Tubular Device Size

### General Device Size

The device has a size small enough to fit, when folded, in a standard drug capsule, and large enough to contain the desired drug formulation amount, and, when released from the capsule at the targeted location in the GI tract (e.g., human small intestine), to expand to a size that enables it to be positioned coaxially in the intestine (i.e., with the longitudinal axes of the device and the intestine parallel to each other) (FIG. 1).

The presence of voids (e.g., holes) in the generally tubular structure of the device of the present disclosure (see FIGS. 2A-F) allows it to have a volume sufficiently small, when folded, to fit in the capsule. This void can take the form of mesh holes in the tube wall, mesh holes in the foldable guiding elements, tube lumen and/or of foldable guiding elements, etc.

Generally, the diameter of the small intestine of a healthy adult ranges between 2.0 and 3.5 cm, the average for an 8-year-old child is about 2.1 cm, and the average for a 15-year-old child is about 2.3 cm. The average diameter of the small intestine of a healthy medium size dog or a similar sized animal is between 0.8 and 1.5 cm. The diameter of standard two-piece capsules used for oral drugs ranges from ˜0.5 to 1 cm, and their locked length (i.e., when the two pieces are assembled) ranges from ˜1.2 to 2.8 cm, preferably from about 2 cm to about 2.8 cm, more preferably from about 2.1 cm to about 2.8 cm. The capsules internal volume ranges between about 0.13 to about 1.4 mL, preferably from about 0.4 mL to about 1.4 mL.

Without being so limited, the device's folded volume ranges from about 0.13 mL to about 1.4 mL, preferably from about 0.4 mL to about 1.4 mL

Without being so limited, the device's expanded diameter (from its axis to its structural element that is the furthest away from its axis (i.e., the device's outer peripheral surface or, the case being, the opening rims of drug cavities erected above the device's outer peripheral surface) is from about 0.45 cm to about 6 cm. When the device is used for a human adult or child, the device's diameter preferably ranges from about 1.1 cm to about 6 cm. When the device is used for medium size dogs or other similar sized animals, the device's diameter preferably ranges from 0.45 cm to 2.4 cm.

In specific embodiments, the device's expanded diameter is between about 0.45 cm to about 6 cm, about 0.8 cm to about 6 cm, about 0.9 cm to about 6 cm, about 1 cm to about 6 cm, about 1.1 cm to about 6 cm, about 1.2 cm to about 6 cm, about 1.3 cm to about 6 cm, about 1.4 cm to about 6 cm, about 1.5 cm to about 6 cm, about 2 cm to about 6 cm, about 2.5 cm to about 6 cm, about 3 cm to about 6 cm, about 3.5 cm to about 6 cm, about 4 cm to about 6 cm, or about 4.5 cm to about 6 cm; about 0.45 cm to about 5 cm, about 0.8 cm to about 5 cm, about 0.9 cm to about 5 cm, about 1 cm to about 5 cm, about 1.1 cm to about 5 cm, about 1.2 cm to about 5 cm, about 1.3 cm to about 5 cm, about 1.4 cm to about 5 cm, about 1.5 cm to about 5 cm, about 2 cm to about 5 cm, about 2.5 cm to about 5 cm, about 3 cm to about 5 cm, about 3.5 cm to about 5 cm, or about 4 cm to about 5 cm; about 0.45 cm to about 4.5 cm, about 0.8 cm to about 4.5 cm, about 0.9 cm to about 4.5 cm, about 1 cm to about 4.5 cm, about 1.1 cm to about 4.5 cm, about 1.2 cm to about 4.5 cm, about 1.3 cm to about 4.5 cm, about 1.4 cm to about 4.5 cm, about 1.5 cm to about 4.5 cm, about 2 cm to about 4.5 cm, about 2.5 cm to about 4.5 cm, about 3 cm to about 4.5 cm, about 3.5 cm to about 4.5 cm, about 0.45 cm to about 4 cm, about 0.8 cm to about 4 cm, about 0.9 cm to about 4 cm, about 1 cm to about 4 cm, about 1.1 cm to about 4 cm, about 1.2 cm to about 4 cm, about 1.3 cm to about 4 cm, about 1.4 cm to about 4 cm, about 1.5 cm to about 4 cm, about 2 cm to about 4 cm, about 2.5 cm to about 4 cm, about 3 cm to about 4 cm, or about 3.5 cm to about 4 cm; about 0.45 cm to about 3.5 cm, about 0.8 cm to about 3.5 cm, about 0.9 cm to about 3.5 cm, about 1 cm to about 3.5 cm, about 1.1 cm to about 3.5 cm, about 1.2 cm to about 3.5 cm, about 1.3 cm to about 3.5 cm, about 1.4 cm to about 3.5 cm, about 1.5 cm to about 3.5 cm, about 2 cm to about 3.5 cm, about 2.5 cm to about 3.5 cm, or about 3 cm to about 3.5 cm; about 0.45 cm to about 3 cm, about 0.8 cm to about 3 cm, about 0.9 cm to about 3 cm, about 1 cm to about 3 cm, about 1.1 cm to about 3 cm, about 1.2 cm to about 3 cm, about 1.3 cm to about 3 cm, about 1.4 cm to about 3 cm, about 1.5 cm to about 3 cm, about 2 to about 3 cm, about 2.5 cm to about 3 cm, about 0.45 cm to about 2.5 cm, about 0.8 cm to about 2.5 cm, about 0.9 cm to about 2.5 cm, about 1 cm to about 2.5 cm, about 1.1 cm to about 2.5 cm, about 1.2 cm to about 2.5 cm, about 1.3 cm to about 2.5 cm, about 1.4 cm to about 2.5 cm, about 1.5 cm to about 2.5 cm, about 2 to about 2.5 cm; about 0.45 cm to about 2.4 cm, about 0.8 cm to about 2.4 cm, about 0.9 cm to about 2.4 cm, about 1 cm to about 2.4 cm, about 1.1 cm to about 2.4 cm, about 1.2 cm to about 2.4 cm, about 1.3 cm to about 2.4 cm, about 1.4 cm to about 2.4 cm, about 1.5 cm to about 2.4 cm, about 2 to about 2.4 cm, etc.).

Without being so limited, the ratio of the diameter of the expanded device over the subject's small intestine diameter ranges from about 0.3 to about 3. In a more specific embodiment, it ranges from about 0.3 to 1, from about 0.3 to less than about 1, from about 0.3 to about 0.95, from about 0.3 to about 0.9, from about 0.3 to about 0.85, or from about 0.3 to about 0.8. In a more specific embodiment, it ranges from about 0.4 to 1, from about 0.4 to less than about 1, from about 0.4 to about 0.95, from about 0.4 to about 0.9, from about 0.4 to about 0.85, or from about 0.4 to about 0.8. In a more specific embodiment, it ranges from about 0.5 to 1, from about 0.5 to less than about 1, from about 0.5 to about 0.95, from about 0.5 to about 0.9, from about 0.5 to about 0.85, or from about 0.5 to about 0.8. In a more specific embodiment, it ranges from about 0.6 to 1, from about 0.6 to less than about 1, from about 0.6 to about 0.95, from about 0.6 to about 0.9, from about 0.6 to about 0.85, or from about 0.6 to about 0.8. In a more specific embodiment, it ranges from about 0.7 to 1, from about 0.7 to less than about 1, from about 0.7 to about 0.95, from about 0.7 to about 0.9, from about 0.7 to about 0.85, or from about 0.7 to about 0.8. In another embodiment, it ranges from about 0.7 to about 1.3.

Without being so limited, the device's expanded length is from about 2 cm to about 6 cm. Without being so limited, the device's expanded “lumen” is from about 0.4 cm to about 4.5 cm. When the device is a simple foldable tubular mesh (e.g., FIGS. 2A-2C), its lumen diameter, corresponds to the tube's lumen diameter. When the device comprises foldable guiding members (e.g., wings, hollow prisms) radially extending from a central tube or rod, its “lumen” diameter corresponds to the diameter from the internal wall of its outer peripheral surface to the external wall of central tube/rod.

Advantageously, the expanded state diameter and size is known and controlled.

The dimension of the device can be easily adjusted according to the drug dose, composition of the formulation and patient needs. The dimension of each feature could be adjusted within a range. For example, if a single tubular mesh is used e.g., as shown in FIGS. 2A-C, a wall thickness of about 0.5 mm-4 mm can be used.

### Drug Cavity Sizes

The tunable drug cavities are sized so as to allow a simple drug loading step such as coating (e.g., dip, spray coating), drop casting or to allow insertion of minitablets therein (e.g., diameter of 1 mm-3 mm) and/or to allow delivery over a period of between 5 minutes up to 6 hours.

Each cavity can have a volume of at least 10 nL, at least 20 nL, at least 30 nL, at least 40 nL, at least 50 nL, at least 60 nL, at least 70 nL, at least 80 nL, at least 90 nL; at least 0.1 μL, at least 0.2 μL, at least 0.5 μL, at least 1 μL, at least 5 μL, at least 10 μL, at least 20 μL, at least 30 μL, at least 50 μL, at least 60 μL, at least 70 μL and up to 100 μL; between 10 nL and 100 μL; or between 20 nL and 100 μL; or between 30 nL and 100 μL; or between 40 nL and 100 μL; or between 50 nL and 100 μL; or between 60 nL and 100 μL; or between 70 nL and 100 μL; or between 80 nL and 100 μL; or between 90 nL and 100 μL; or between 0.1 μL and 100 μL; or between 0.2 μL and 100 μL; or between 0.5 μL and 100 μL; or between 1 μL and 100 μL; or between 5 μL and 100 μL; or between 10 μL and 100 μL; or between 20 μL and 100 μL; or between 30 μL and 100 μL; or between 50 μL and 100 μL; or between 60 μL and 100 μL; or between 70 μL and 100 μL. The device can comprise a combination of cavities having different sizes as listed above or within the stated ranges.

### Device Elasticity

Elasticity is the ability of a body to resist a distorting influence and to return to its original size and shape when that influence or force is removed. The device's foldable elements' elasticity enables it to be folded and inserted into a capsule, and once released from the capsule in the GI tract, enables it to self expand to regain its original size and shape (expanded state). The device has an elasticity that enables its foldability and self-expandability.

In specific embodiments, the device material has a Young's modulus of about 0.01 MPa to about 1000 MPa (about 0.1, 0.2, 0.5, 1, 2, 3, 4, 5, 6, 7, 10, 15, 20, 30, 40, 50, 75 MPa, to about 50, 100, 200, 300, 400, 500, 600, 700, 800 or 900 MPa) at a temperature of between about 20° C. and about 55° C.

### Device Material

The material of the device is pharmaceutically acceptable. It is also sufficiently flexible/elastic to allow for its easy mechanical folding and expanding. The device material comprises or consists of an elastomer. The device material is such as to confer it has Young's modulus of about 0.01 MPa to about 1000 MPa. Without being so limited, materials encompassed by the present disclosure include elastomers e.g., polyester-based elastomers, such as poly(&-caprolactone), poly(glycolide), poly(lactide), poly(hydroxyalkanoate), poly(4-hydroxybutyrate), poly(dioxanone), poly(1,3-trimethylene carbonate), poly(ethylene succinate), polybutylene terephthalate, and their copolymers; polyether-based elastomers, such as poly(ethylene glycol), poly(tetramethylene)glycol, poly(propylene oxide), poly(p-phenylene oxide), polyaryletherketone, polyether ether ketone, polyphenyl ether, polytetrahydrofuran, and their copolymers; polyamides-based elastomers; polyurethanes-based elastomers; poly(ester amide)-based elastomers; polysaccharides-based elastomers; poly(β-thioester)-based elastomers, poly(β-amino ester)-based elastomers, poly(β-thioether ester)-based elastomers; polydimethylsiloxane (PDMS)-based elastomers; polyacrylates-based elastomers; synthetic and natural rubbers such as polybutadiene; copolymers (block, diblock, or triblock) of at least two thereof; or a mixture of at least two of these materials. In specific embodiments, the material is non biodegradable (e.g., polyether-based elastomers; polydimethylsiloxane (PDMS)-based elastomers; polyacrylates-based elastomers; synthetic and natural rubbers such as polybutadiene). In other specific embodiments, the material is biodegradable (e.g., polyester-based elastomers, polyamides-based elastomers; polyurethanes-based elastomers; poly(ester amide)-based elastomers; polysaccharides-based elastomers; poly(β-thioester) and poly(β-amino ester)-based elastomers). When these polymers are chemically functionalized to form a cross-linked network, the main chain of the polymers could be degraded under different mechanisms such as hydrolysis, reverse reaction, chemical cleavage, enzymatic degradation, etc. The degradation kinetics could also be adjusted from a few hours to a few days under the intestinal fluid conditions.

The device can be made of a single material or multiple materials, e.g., a first material for foldable parts thereof (single tube or foldable guiding members) and a second material for the central tube or rod. The device material(s) may be biodegradable or nonbiodegradable. In a more specific embodiment, it is biodegradable. Degradable elastomers degrade under physiological conditions with tunable kinetics. Their degradability minimizes risks associated with congestion in the GI tract. In specific embodiments, elastomeric materials based on polyesters, or poly(β-amino ester) s, which are compatible with both 3D printing and molding fabrication methods are used. In specific embodiments, the device does not include a metal.

In specific embodiments, the device may be produced by 3D printing or molding.

### Capsule Material

Any pharmaceutically acceptable capsule can be used for devices of the present disclosure. The capsule can advantageously be enteric to enable its disintegration in the desired location of the GI tract, e.g., the small intestine. Without being so limited, capsules that can be used for the present disclosure can be made from gelatin, hydroxypropylmethyl cellulose or pullulan and their derivatives. The capsules can be intrinsically enteric or coated with an enteric polymer such as methacrylic acid copolymers, cellulose acetate phthalate, hydroxypropylmethylcellulose phthalate, or polyvinyl acetate phthalate. As used herein the term “enteric capsule” refers to a capsule made from an enteric polymer and/or a capsule coated with an enteric coating.

### Drug

Drug(s) as used herein refers to any pharmacologically active molecule or mixture of molecules (including non approved active pharmaceutical ingredients (APIs)). As used herein, mixtures of molecules display pharmacological activity. Without being so limited, biological extracts such as plant or animal extracts are encompassed by the term mixture of molecules, and thereby by the term drug. Drugs that can be incorporated into the device of the present disclosure can be low molecular weight APIs (<1000 Da) or large molecules (>1000 Da) such as polysaccharides, peptides, proteins, nucleic acids, dendrimers, polymer-drug conjugates, proteolysis-targeting chimeras (PROTACs); Lysosome-targeting chimaeras (LYTAC); antibody-based PROTACs (AbTAC), antibody-drug conjugates, lipid or polymeric nanoparticles, exosomes, and vaccine antigens. While devices of the present disclosure are most advantageously used to deliver drugs with high molecular weight (without being so limited, e.g., between 1 kDa and 50 kDa; e.g., about 1 kDa to about 45 kDa) or size (without being so limited between 0.5 nm and 10 nm; or between about 0.5 nm and 6 nm) as such drugs typically display low oral absorption and require injection, there are no lower size limitations to drugs that can be used in the devices of the present disclosure. The devices of the disclosure would also advantageously be used for drugs displaying low oral bioavailability related to degradation in the GI tract and/or poor diffusibility (poor absorption) across the mucosa, including drugs smaller than 1 kDa, and including cannabis oils including e.g., cannabidiol and/or cannabigerol and/or tetrahydrocannabinol.

Drugs that would particularly benefit from the delivery device of the present disclosure include the following: (1) semaglutide, an anti-diabetic and anti-obesity medication that acts like human glucagon-like peptide-1. The drug has a molecular weight of 4113 Da with an oral bioavailability ˜1% even in the presence of large amount of permeation enhancers as excipients. The recently approved oral tablet (Rybelsus) from Novo Nordisk for diabetes) also requires the drug to be taken at a specific time before breakfast every day, as the absorption is affected by food in the stomach. (2) octreotride: has an oral bioavailability of 0.5-1% even in the presence of permeation enhancers; (3) human growth hormone, a drug to treat children's growth disorders and adult growth hormone deficiency. As is typical of protein drugs with a high molecular weight (22 kDa) and being prone to be degraded in the GI tract, there are currently no oral formulations of this drug on the market; (4) insulin is currently only available for injection or pulmonary administration; (5) TNF-alpha inhibitors are antibodies that need to be injected. Other peptide and protein (macromolecular drugs) drugs with low oral bioavailability and high sensitivity to degradation in the GI-tract: 6) PROTACs, a new type of drug molecule that can degrade the target pathogenic proteins and regulate the related signaling pathways. With similar molecular weight to peptide-based drugs, the PROTACs often have low oral bioavailability. The device could avoid several modifications to achieve stability and absorption via the GI tract; and (7) vaccines: As injected vaccines only trigger systemic immunization; mucosal immunity could also or alternatively be stimulated via the transmucosal route.

In view of the size limitation of the device, and consequently limited drug compartment(s) size, the device may be used to deliver drugs that can usefully be administered at a dose lower than about 100 mg (or lower than about 50 mg)

### Drug Formulation

The drug(s) can be loaded in the device with at least one excipient (one or more excipient(s)).

Without being so limited, excipients/carriers can be loaded inside one or more compartment(s) (cavities) of the device, included as filler in the capsule enclosing the device and/or coated on the capsule.

Without being so limited, one or more of the following excipients can be used in addition to the drug:

Enteric coating: In order to release the drug delivery device in the intestine, unless a capsule made of an enteric polymer is used, enteric coating avoids premature opening of the capsules in the stomach. Furthermore, by tuning the thickness of the coating layer as well as the pH sensitivity of the enteric polymer, it is also possible to control the time and location of the release. As indicated above, the enteric material can be coated on capsules enclosing the device of the present disclosure.

Polymeric and lipidic excipients: One or more polymeric/lipidic excipients can be used in order to control its release kinetics in the GI tract and/or create bulk volume. Without being so limited, these excipients can be poly(vinyl alcohol) (PVA), poly(N-vinylpyrrolidone) (PVP), glycerol, poly(ethylene glycol) (PEG), polycarbophil, starch and derivatives, fatty acids, polysaccharides (e.g., cellulose, chitosan, cyclodextrins, derivatives thereof) carbomer, alginate and fatty acids derivatives, and waxes. The one or more polymeric/lipidic excipients can be incorporated inside one or more cavity (with or separately from the drug(s) on the device outer peripheral surface, or as a filler inside the capsule with the device. Without being so limited, about 0.5 mg-500 mg of polymeric/lipidic excipients could be included in the device or capsule of the present disclosure.

Enzymatic inhibitors: A major obstacle to the delivery of peptides is their susceptibility to enzymatic degradation especially for peptide and protein-based drugs. Enzymatic inhibitors such as protease inhibitors that can inactivate luminal and/or brush border enzymes have therefore been proposed as the excipient for oral drug delivery. Protease inhibitors that can be used in the device include without being so limited, citric acid, aprotinin, Bowman-Birk inhibitor; bacitracin and derivatives; N-acetyl-cysteine; soybean trypsin inhibitor; camostat mesylate; sodium glycocholate;

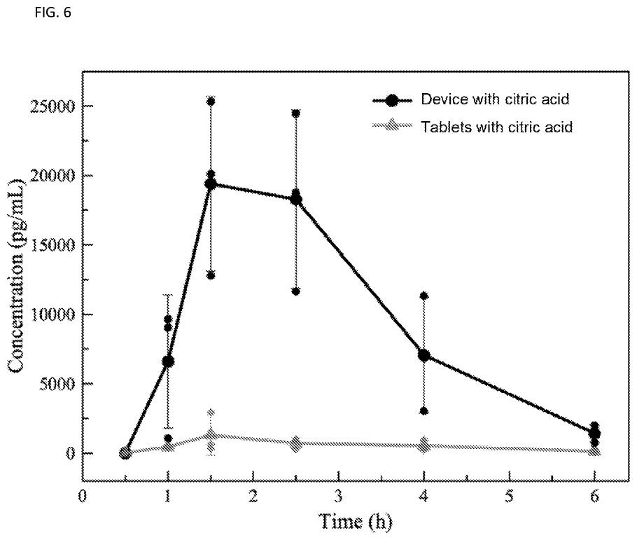

hydroxyethylcellulose; and poly(acrylic acids). (See e.g., FIG. 6). One or more protease inhibitor(s) can be incorporated inside one or more cavity (with or separately from the drug(s) on the device outer peripheral surface, or as a filler inside the capsule with the device. Without being so limited, about 0.1 mg-800 mg (in a specific embodiment 0.1 mg to 775 mg, to 750 mg, to 700 mg, to 650 mg, to 600 mg or to 500 mg) of enzymatic inhibitors such as protease inhibitors could be included in the device or capsule of the present disclosure.

Adjuvants: adjuvants such as but not limited to squalene, aluminum and 3-deacyl-monophosphoryl lipid A can be used. One or more adjuvants(s) can be incorporated inside one or more cavity (with or separately from the drug(s)) on the device outer peripheral surface, or as a filler inside the capsule with the device. Without being so limited, about 0.5 mg-500 mg of adjuvant could be included in the device or capsule of the present disclosure.

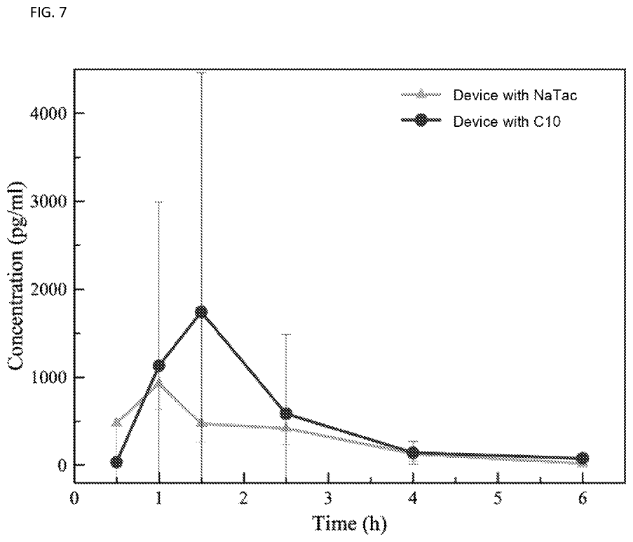

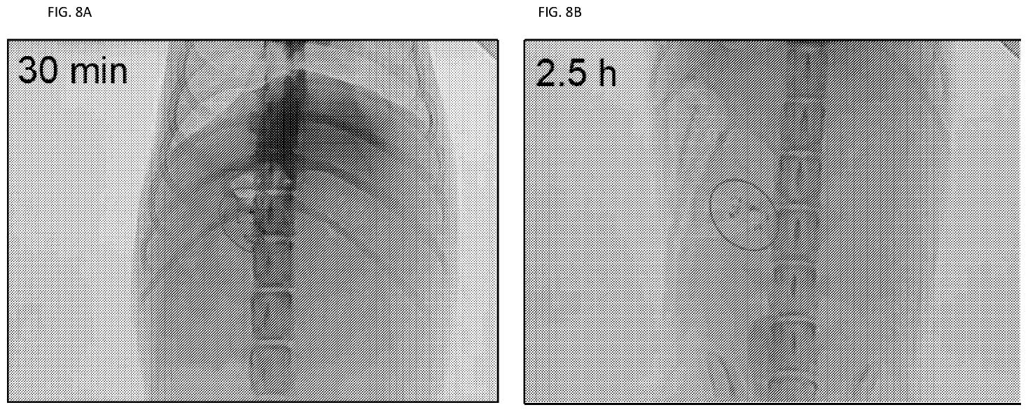

Permeation enhancers: They are compounds capable of enhancing membrane permeability in the GI tract. They function with different mechanisms of actions, e.g., by disrupting the mucus layer, transiently opening tight junctions, or fluidizing the epithelial cell membranes. The combination of the device with the chemical disruption produced by a permeation enhancer can further promote the diffusion of the drug (e.g., hydrophilic macromolecules) (see e.g., FIG. 7).

As used herein, the permeation enhancers that can be used in the device or capsule include without being so limited, salcaprozate sodium (SNAC); fatty acids and derivatives, such as sodium caprate (C10), sodium caprylate, and palmitoylcarnitine; bile salts and other steroidal detergents, such as deoxycholate, sodium taurocholate (NaTaC), glycocholate, and saponin; natural and synthetic surfactants, such as phospholipids, sodium lauryl sulphate, dioctyl sodium sulfosuccinate, and derivatives thereof; chelators such as EDTA, citric acid/citrate, and polyacrylates; positively charged polymers such as chitosan; cyclodextrins and derivatives; laurocapram and derivatives; thiomenthol and derivatives; and polysorbates and derivatives. In a specific embodiment, the permeation enhancer is C10. The one or more permeation enhancers can be incorporated inside one or more cavity (with or separately from the drug(s)) on the device outer peripheral surface, or as a filler inside the capsule with the device. Without being so limited, about 0.5 mg-500 mg of permeation enhancer could be included in the device or capsule of the present disclosure.

The total weight of drug and of the one or more excipient(s) in the capsule (e.g., in the device, and/or as filler) is about 100 mg to 3 g.

The device of the present disclosure can migrate throughout the GI tract. Hence, in specific embodiments, the device does not contain an adhesive agent (e.g., to hold the expanded outer peripheral surface in position in the GI tract during delivery of the drug through the intestinal wall).

### Device Operation

The present disclosure provides for the use of a swallowable and self-expandable drug delivery device described herein for the non-parenteral systemic delivery of a drug in a subject in need thereof. The device is enclosed in an enteric capsule to ensure targeted drug delivery in the intestine. After it is ingested, the capsule follows normal transit and once it reaches its destination in the intestine, it releases the device, and optional filler comprising one or more excipients. The release of the device allows it to self-expand through unfolding and to position itself coaxially in the intestine. The device's expansion is not caused by being inflated i.e., it does not rely on a distention through the device being filled with air or gas (balloon effect) as a result of e.g., a chemical reaction. The device's expansion is not either dependent on the presence of biological matter in the GI tract. The expansion is triggered by the self-unfolding of the device upon its release from the capsule. This unfolding is caused by the elasticity of the material composing the device. The central void spaces created or amplified by this unfolding within the outer peripheral surface of the device (e.g., by the device's mesh structure) and/or between the outer peripheral surface of the device and its central axis, may advantageously allow food and fluid transit across the device and trough the GI. The device does not seal the GI tract upon its expansion. The device then follows the normal transit while releasing the at least one drug(s) and excipient(s) that it contains, towards and in close proximity to the intestinal epithelium or touching it. In specific embodiments, the expanded device remains smaller than the diameter of the intestinal lumen and does not press against the intestinal mucosa. A steep drug concentration gradient can thus be established and maintained to achieve better drug permeation.

The delivery of the drug from the device through the subject's intestinal epithelium and into the subject's systemic circulation (via lymphatic vessels and/or blood vessels) occurs through passive migration of the drug if the latter is not linked to a ligand capable of recognizing a receptor at the surface of the epithelial mucosa. Since after the device release from the capsule, the at least one drug loaded cavity faces the intestinal epithelium and touches or is in close proximity thereto throughout the device transit, the drug release towards the epithelium is always favored (“unidirectional release”). According to the present disclosure, the device is dynamic (i.e., moves with normal transit once orally administered and throughout drug delivery). It is a needle free, electricity free delivery that does not involve physical piercing of the epithelium.

### Combination Therapies

The device of the present disclosure can be applied as a stand-alone delivery means for drugs or in combination with other drug delivery strategies, such as permeation enhancers as described above.

### Route

The device is for oral delivery.

### Subject

As used herein the term “subject” is meant to refer to any animal, such as a mammal, including a human, dog, cat, pig, cow, monkey, cattle, horse, etc. In a particular embodiment, it refers to a human, in specific embodiments to an adult subject.

### Device Manufacturing Method

The device of the present disclosure can be designed using common Computer Aided Design (CAD) softwares, such as AutoCAD™, Solidworks™, etc. The devices can be produced using any known method, including but no limited to 3D printing with techniques such as digital light processing (DLP), carving, molding (e.g., compression molding, mold casting, and injection molding), which enables rapid prototyping, optimization, as well as mass production.

### Drug Loading

Any known drug loading method can be used including but not limited to coating (e.g., dip, film formation, spray coating), drop casting, paste injection, mini-tablet loading (e.g., diameter of 1 mm-3 mm), etc. With the film formation method, the drugs and excipients are dissolved or dispersed in a suitable solvent in the absence of presence of other excipients (e.g., lipids, polymers), which is then evaporated to leave a solid matrix in the device cavities. When the film formation is conducted by drug dispersion in a lipid matrix, the lipidic matrix can be heated, deposited on the device (e.g., cavities) and cooled down. In the paste injection method, a semi-solid solution or dispersion of the drug and excipients is deposited by extrusion in the device.

### Drug Delivery

Once the drug loaded device is loaded in the capsule, and the capsule is swallowed by the patient, the capsule transits to its delivery target location in the GI tract (e.g., small intestine), releases the device, which then unfolds and positions itself coaxially in the small intestine lumen, with drug compartments opening in close proximity to the GI epithelium or touching it. Without being so limited, in Examples presented herein, the drug crosses the epithelium, and reaches the blood and/or lymphatic vessels (systemic circulation).

Self-expandable: the elastomeric materials and the design of the device allow the device to self-expand upon its release from the capsule without the need for any actuation mechanisms, such as chemical reaction or heat.

The aspect ratio (diameter and length) of the device also ensures its correct positioning in the GI tract.

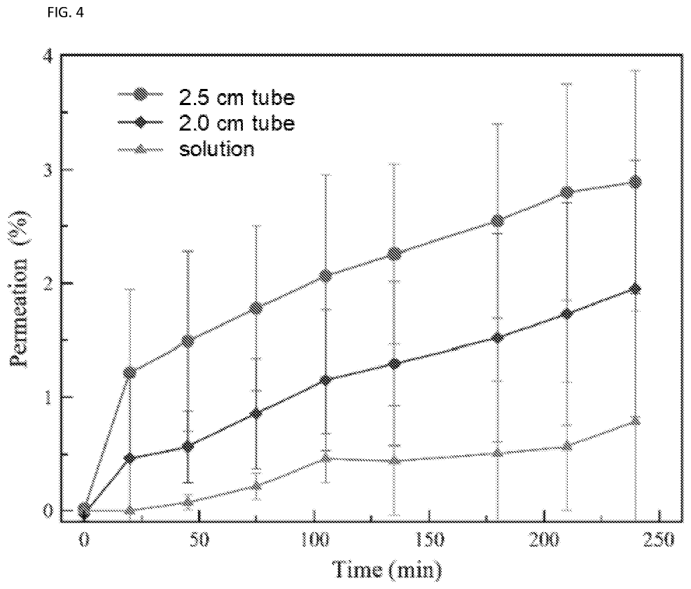

Unidirectional release and sharp drug concentration gradient: Due to the highly dynamic GI tract environment, controlling the release direction of the drug in vivo is highly challenging. The expandable device of the present disclosure (tubular structure) promotes the correct orientation of drug towards the intestinal epithelium and thus achieves localized high drug concentration which promotes diffusive flux of the drug. According to Fick law of diffusion, diffusive flux goes from a high-concentration area to a low-concentration area and is proportional to the concentration gradient. Ex vivo studies were performed on excised pig intestines (FIG. 4) to examine such effect. A considerable increase in intestinal drug permeation was observed upon application of the expandable device with a diameter comparable to that of the intestine, compared to a smaller device and a solution of the drug.

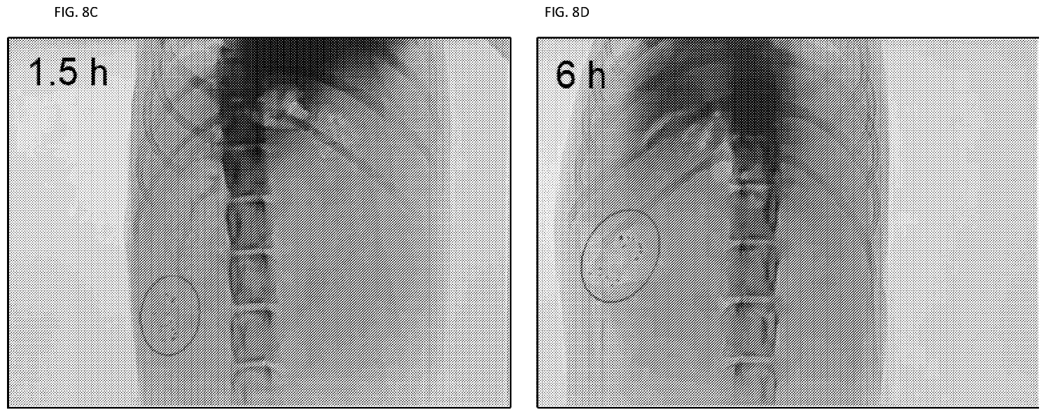

Non-invasive: The expandable device is non-invasive and does not require any strong physical disruption of the intestinal tissue. This may reduce potential safety risks due to the systemic exposure to food antigens and microorganisms. Furthermore, with a tubular structure the device avoids or reduces disturbing the passage of other components in the intestine, such as liquids and foods. Repetitive dosing to beagle dogs for a duration of almost a year has never demonstrated any signs of congestion of the GI tract or other side effects such as weight loss. In fact, in vivo Xray imaging (FIG. 8) has shown that the device could transit through the GI tract smoothly and reach colon within six hours after administration.

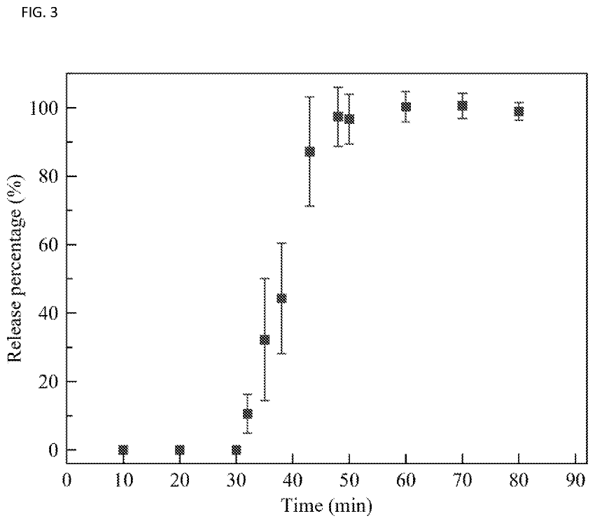

Versatile for different drug formulations: The swallowable device is usable for the delivery of various types of drugs and excipients. Various types of excipients can be loaded in the delivery device together with the drug compounds, such as permeation enhancers (e.g., polymer matrix) (e.g., permeation enhancers such as C10 (e.g., FIG. 7), polymers, and enzymatic inhibitors (e.g., protease inhibitors such as citric acid (see e.g., FIG. 6)). A controlled release kinetics can thus be achieved (see e.g., FIG. 3). With a tunable drug compartment design, the loaded doses are also easily adjusted.

Robustness: A common disadvantage of the physical mixture of drugs and other excipients is the lack of robustness in achieving high drug concentration near intestinal wall. For example, with the commercial semaglutide drug tablet, the rate of successful delivery is less than 50% for single dosing. It was reported that in the single-dose trial, a high proportion of subjects distributed among all oral semaglutide treatment arms had no measurable semaglutide in plasma (Granhall et al., 2019). In contrast, the expandable device ensures a close contact of the drug and mucosa and thus leads to more reproducible delivery profile.

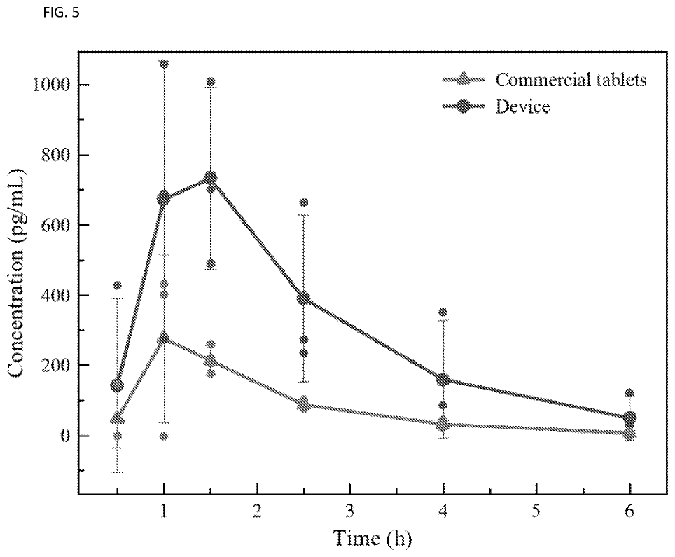

Higher bioavailability: The bioavailability of most oral formulations for macromolecular drugs such as peptides is very low, i.e., 1% or lower. A series of in vivo experiment in beagle dog models were performed using desmopressin (Mw 1069 Da, antidiuretic peptide) as the model drug. The self-expandable drug delivery device could achieve ˜3.6 times higher bioavailability than commercial tablets (FIG. 5). When combined with citric acid, the optimized formulation led to more than 17 times higher absorption (FIG. 6) than commercial tablets that were administered together with citric acid, or more than 107 times higher compared to commercial tablets used without citric acid. Of note, most of the approved oral peptide drugs have a bioavailability less than 1%.

Patient friendliness and reduced health care cost. The device application is simple and user friendly as no special training or other devices are required. Therefore, drugs that typically must be administered via injections can be pain-free self-administrated by patients, which most likely increases patient acceptance and hence their compliance, especially for children or subjects with antipathy to needles. The fact that the device is self-applicable allows the home administration of drugs, which further increases patient comfort as well as reduces the costs and requirements for medical staff.

Scalability and costs. Due to the simple and scalable manufacturing process by molding, pre-existing manufacturing plants can be used with small adjustments. The device does not rely on external devices such as iontophoresis, electronic circuitry, electrodes, inflation means, thereby minimizing the device manufacturing costs. Moreover, the drug loading processes by e.g., film formation or paste injection, are well established processes and can be easily adapted. As a result, simple manufacturing, low-cost materials and simple implementation in existing manufacturing plants make the technology readily available for small and large companies.

More specifically, in accordance with the present disclosure, there are provided the following items:

Item 1. Swallowable, foldable and self-expandable oral drug delivery tubular device, the device:
comprising an outer peripheral surface;
having an expanded length of about 2 cm to about 6 cm;
having an expanded diameter of about 0.45 cm to about 6 cm; and
comprising at least one cavity enclosing a drug, the at least one cavity facing outward from the outer peripheral surface of the device.

Item 2. The device of item 1, wherein the tubular device comprises a single tubular mesh.

Item 3. The device of item 1, wherein the tubular device comprises foldable guiding members radially extending from a central tube or rod, each foldable guiding member comprising a guiding member outer peripheral surface, the combined guiding member outer peripheral surfaces of all foldable guiding members forming the device's outer peripheral surface.

Item 4. The device of any one of items 1-3, wherein the drug is in a formulation further comprising at least one excipient.

Item 5. The device of item 4, wherein the at least one excipient comprises a permeation enhancer.

Item 6. The device of item 4 or 5, wherein the at least one excipient comprises a protease inhibitor.

Item 7. The device of any one of items 1-6, wherein the drug has a molecular weight of about 1 kDa to about 50 kDa.

Item 8. The device of any one of items 1-6, comprising drug cavities having a volume between 10 nL and 100 μL.

Item 9. The device of any one of items 1-8, wherein the device material comprises or consists of an elastomer.

Item 10. Enteric capsule enclosing the device defined in any one of items 1-9.

Item 11. The capsule of item 10, further comprising a filler. In a specific embodiment, the capsule further comprises f a further excipient.

Item 12. The device of any one of items 1-9 or the capsule of item 10 or 11, for oral administration of the drug to a subject.

Item 13. The device of capsule of item 12, wherein the subject is a human and the device's expanded diameter is of about 1.1 cm to about 6 cm.

Item 14. A use of the device as defined in any one of items 1 to 9 enclosed in an enteric capsule, for the oral administration of the drug to a subject, the use comprising:
orally administering the capsule to the subject, whereby when the capsule releases the device in the small intestine,
the device self-expands and positions itself coaxially in the intestine, and whereby the drug migrates through the intestine epithelium and enters systemic circulation of the subject.

Item 15. The use of item 14, wherein the subject is a human.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

### In the Appended Drawings:

FIG. 1: Illustration of an exemplary self-expandable oral drug delivery device operation.

FIGS. 2A-F: Illustrative embodiments of the self-expandable oral drug delivery device.

FIG. 3: In vitro drug release in simulated intestinal fluid by the self-expandable oral drug delivery device.

FIG. 4: Comparison of the ex vivo effects of two diameter sizes (2 cm and 2.5 cm) of self-expandable oral drug delivery device on transmucosal drug delivery.

FIG. 5: Comparison of the plasma concentration of drug in beagle dogs after administration of a desmopressin loaded self-expandable oral drug delivery device with that of Minirin™ tablets.

FIG. 6: Comparison of the plasma concentration of drug in beagle dogs after administration of a desmopressin loaded self-expandable oral drug delivery device in combination with citric acid with that obtained with Minirin™ tablets in combination with citric acid.

FIG. 7: Comparison of the plasma concentration of drug in beagle dogs after administration of a desmopressin loaded self-expandable oral drug delivery device in combination with two different permeation enhancers, C10 and NaTaC.

FIGS. 8A-D: Representative in vivo imaging of the device transit through GI tract of beagle dogs.

### DESCRIPTION OF ILLUSTRATIVE EMBODIMENTS

The present disclosure is illustrated in further details by the following non-limiting examples.

### Example 1: Device Preparation

### Device Design

The expandable devices were designed with 3D Computer Aided Design (CAD) software SolidWorks™ and those used in Examples 3 to 9 are tubular mesh devices as shown in FIG. 2A and have the following dimensions:

| Example (FIG. #) | Expanded diameter | Expanded length |
|---|---|---|
| 4 (FIG. 3) | 8 mm | 2.5 cm |
| 5 (FIG. 4) | 2 cm; 2.5 cm | 3 cm |
| 6 (FIG. 5) | 8 mm | 2.5 cm |
| 7 (FIG. 6) | 8 mm | 2.5 cm |
| 8 (FIG. 7) | 8 mm | 2.5 cm |
| 9 (FIG. 8A-B) | 8 mm | 3 cm |

### Device Preparation

To prepare a photopolymerizable resin, a poly(β-thioether ester) polymer was prepared by copolymerizing (ethylenedioxy) diethanethiol (EDT) and 1,4-butanediol dimethacrylate (BDA) yielding the poly(β-thioether ester) polymer with an averaged molecular weight of 6300 Da.

To manufacture the devices used in the Examples herein, the polymer (9 g), a phosphine oxide initiator (0.75 wt %) (phenylbis(2,4,6-trimethylbenzoyl) phosphine oxide (BAPO)) a photo absorber Sudan I (0.04 wt %), the dilution solvent N-Vinylpyrrolidone (NVP) (11 wt %), and UV inhibitor vitamin E (0.3 wt %) were mixed and sonicated at 40° C. until a homogeneous resin was obtained.

A commercial DLP 3D printer (Asiga PICO2, Sydney, Australia) was employed to build the device. The printer comprised a LED light source of 405 nm and a customized tray with a heating system to allow printing at various temperatures e.g., 75° C. After printing, resin residuals were removed with isopropanol and ethanol. Subsequently, the printed device was cured under 405 nm UV-light for 15 min followed by extensive rinsing with ethanol and drying overnight.

### Example 2: Illustrative 3D Structures of the Device

The expandable drug delivery device can have various 3D structures provided they have the characteristics listed above. FIGS. 2A-F present specific embodiments of the swallowable self-expandable device of the present disclosure. FIGS. 2A-C present devices that consist of simple tubular meshes comprising drug cavities on their outer peripheral surface (external tubular wall). Their top views show that they are hollow. In FIG. 2A, a side view of the tubular mesh is shown where 6 shallow cup-shaped drug cavities can be observed (i.e., other drug cavities are present on sides of the device that are not visible on the view of FIG. 2A) spaced along the external tubular wall (device used in Examples 3-9). In FIG. 2B, a side view of the tubular mesh is shown on which about 14 drug cavities carved in relief on the external tubular wall can be observed which are spaced along the wall in alternance with 14 holes (i.e., other drug cavities and holes are present on sides of the device that are not visible on the view of FIG. 2B). In FIG. 2C, a side view of the tubular mesh is shown that is very similar to that illustrated in FIG. 2A except that the shallow cup-shaped cavities are joined by drug cavities shaped as channels. FIGS. 2D to 2F present devices comprising foldable guiding members radially extending from a central tube, each foldable guiding member comprising an outer peripheral surface containing drug cavities. In FIG. 2D, a top view of the device shows 5 foldable guiding members in the form of hollow trapezoidal prisms. FIG. 2D shows a side view of this device presenting the outer peripheral surfaces of two of these 5 foldable guiding members. A partial view of a third foldable guiding member in also presented. Three shallow cup-shaped drug cavities are shown on each outer peripheral surface. In the devices presented in FIGS. 2E and 2F, the foldable guiding members radially extending from a central tube are wings. In FIG. 2E, a top view of the device shows 6 foldable wings, the free end of each wing being folded so as to form outer peripheral surfaces. In FIG. 2E, a side view of the device shows the outer peripheral surface of one folded wing, comprising 4 shallow cup-shaped drug cavities. This view also partially displays three other wings of the device. In FIG. 2F, a top view of the device shows 5 foldable wings, the free end of each wing being T-shaped so as to form outer peripheral surfaces. In FIG. 2E, a side view of the device shows the outer peripheral surface of one T-shape ended wing, comprising 4 shallow cup-shaped drug cavities. This view also partially displays two other wings of the device.

### Example 3: Drug Loading

A methanol solution containing desmopressin and poly(N-vinylpyrrolidone) (PVP) was first prepared to load the drug formulation onto the devices of Example 1.0.1 mL of the solution was then casted dropwise in the device drug cavities followed by drying overnight to remove all the solvent residues. The device was then loaded in a size 000 gelatin capsule that had been enteric-coated using Eudragit™ L100-55 polymer. Resulting enteric capsules comprising the device loaded with 20 mg of desmopressin and 40 mg of PVP were used in Examples 4 and 5, and resulting enteric capsules comprising the device loaded with 1.2 mg desmopressin and 2.0 mg PVP were used in Examples 6-8, either without any other excipient (Example 6), with 750 mg of citric acid loaded in the capsule (Example 7) or with 4 mg of a permeation enhancer loaded in the capsule (Example 8).

The enteric capsule used in Example 9 contained the device without any drug or excipient.

### Example 4: In Vitro Drug Release

Standard in vitro release studies were performed using USP device type II. The 8 mm diameter expandable device loaded in an enteric capsule as described in Example 3 was first immersed in simulated gastric fluids (SGF, pH=1.2) for half hour (30 minutes) before the solvent was switched to simulated intestinal fluid (SIF, pH=6.8). The temperature was set at 37° C., and the rotating speed was at 100 rpm. Aliquots of 1 mL were sampled at the predetermined time points and the sampled volume was replaced with fresh SGF or SIF buffer.

Quantification was carried by HPLC (VWR Hitachi Chromaster system with 5160 pump, 5260 autosampler, 5310 column oven and 5430 diode array detector, VWR International, Radnor, PA) with the UV wavelength set at 280 nm. The C18 column (XBridge™, 5 μm, 250×4.6 mm, Waters, Milford, MA) was used for the separation, while a mixture of 75% H₂O (0.1% v/v trifluoroacetic acid) and 25% acetonitrile was used for the mobile phase. Results are presented in FIG. 3. At the intestinal pH, desmopressin started being released when the solvent was switched to SIF and was completely released within about 20 minutes. Therefore, with enteric capsules, the device can be protected in the stomach and is only released from the capsule after reaching the intestine. The drug release kinetics is also controlled with the polymeric excipient.

### Example 5: Ex Vivo Studies on the Effects of Localized Drug Delivery by the Self-Expandable Oral Drug Delivery Device

Ex vivo studies were performed on fresh porcine intestines obtained from the slaughterhouse. Intestinal sacs were prepared containing either a desmopressin solution or a 2.0 cm or 2.5 cm diameter expandable device loaded in an enteric capsule as described in Example 3. All intestine tissues were incubated in modified DMEM (i.e., the pH of the medium was adjusted to 6.8 with an HCl solution, and its osmolality was within the range of 280-295 mOsm/kg) for 4 h, during which 1 ml sample solutions were withdrawn at predefined timepoints (0, 20, 45, 75, 105, 135, 180, 210 and 240 min). After filtration, the samples were analyzed by HPLC with the same procedure as that described in Example 4 to quantify the permeation of desmopressin through each sac. Results are presented in FIG. 4. The data shows that the amount of drug that permeated across the epithelium increased when delivered with the expandable device as compared to when administered in the control solution. This shows that the expandable device could promote the transport of drugs through small intestine.

### Example 6: In Vivo PK Study on the 8 mm Diameter Expandable Device

In vivo beagle dog experiments were performed in the Centre National de Biologie Expérimentale, Institute National de la Recherche Scientifique (INRS), Laval, Canada. Procedures involving the care and the use of animals in this study were reviewed and approved by the INRS Institutional Animal Care and Use Committee and performed according to the Canadian Council on Animal Care guidelines and policies. Three beagle dogs with body weights around 10 kg were used. The in vivo experiments were performed using the 8 mm expandable device loaded (for a ratio of the diameter of the expanded device to the diameter of small intestine of about 0.5 to about 1) in an enteric capsule as described in Example 3 containing 1.2 mg desmopressin and 2.0 mg PVP (without any other excipient). As a control formulation, enteric-coated (Eudragit™ L100-55, Essen, Germany) capsules as described in Example 3 containing commercial Minirin® drug tablets (6 pieces (0.2 mg of desmopressin per piece) containing 1.2 mg desmopressin in total) were given orally to the dogs.

After oral administration of the capsules, blood samples were withdrawn at predetermined time points, i.e., 30, 60, 90, 150, 240, and 360 min. Blood samples were collected in K₂EDTA tubes immediately followed by centrifugation at 1700×g for 10 min at 4° C. Subsequently, the plasma was stored in clear polypropylene vials at −80° C. until further analysis. The drug plasma concentration was evaluated using LC-MS/MS method. Briefly, an aliquot of 900 μL dog plasma sample was mixed with 10 μL internal standard (Goserelin; 25 ng/ml) and 300 μL 4% H₃PO₄ acidified water into 1.5 mL Eppendorf tubes. The plasma sample was then extracted using Oasis™ WAX solid-phase extraction cartridges. After extraction, the solution was evaporated under a low vacuum and reconstituted in 120 μL of 11:11:78 acetonitrile/methanol/deionized-water and injected into the Xevo™ G2-XS Accurate Mass Spectrometer. Samples were analyzed in TOF-MRM mode with a quantification limit of <15 μg/mL. Results are presented in FIG. 5. The area under the plasma concentrations vs. time curve (AUC₀₋₆ₕ) for the commercial tablet formulation was 506.21±62.58 pg/mL*h, while the AUC₀₋₆ₕ for the 8 mm expandable device was 1825.7±1055.1 pg/mL*h. The data shows that the expandable device increased the oral bioavailability of desmopressin by about 3.6-fold over that of the commercial tablets.

### Example 7: Effect of the Expandable Device in Combination with a Protease Inhibitor

In vivo experiments were performed on the 8 mm expandable device loaded in a capsule as described in Example 3 containing 1.2 mg desmopressin and 2.0 mg PVP. 750 mg of citric acid was loaded together with the 8 mm diameter device in the capsule. As a control formulation, two enteric-coated (Eudragit® L100-55, Essen, Germany) capsules described in Example 3 but containing, instead of the device, commercial Minirin® drug tablets (3 pieces (0.2 mg per piece) containing 0.6 mg desmopressin in total), and 375 mg citric acid in each capsule was given orally to the dog. Two capsules were used to fit the 750 mg of citric acid. The drug administration and blood sampling and analysis procedure were the same as those described in Example 6. Results are presented in FIG. 6. The area under the plasma concentrations vs. time curve (AUC₀₋₆ₕ) for the expandable device was 54593.4±5497.7 pg/mL*h, while the AUC₀₋₆ₕ for the commercial tablets with citric acid was 3129.4±2180.0 pg/mL*h. This data shows that the expandable device increased the oral bioavailability of desmopressin by about 17.4-fold over that of the commercial tablets in the presence of citric acid. This difference is significant according to a 2-tailed t test and p value of 0.05.

### Example 8: Effect of the 8 mm Expandable Device in Combination with a Permeation Enhancer

In vivo experiments were performed on the 8 mm expandable device and enteric capsule as described in Example 3 containing 1.2 mg desmopressin and 2.0 mg PVP. 4 mg of a permeation enhancer, i.e., one formulation with C10 and the other with NaTaC, was loaded together with the device in the capsule. The drug administration and blood sampling and analysis procedure were the same as those described in Example 6. Results are presented in FIG. 7. The area under the plasma concentrations vs. time curve (AUC₀₋₆ₕ) for the expandable device loaded capsule containing the C10 formulation was 3022.3±4337.1 pg/mL*h, while the AUC₀₋₆ₕ for the expandable device loaded capsule containing the NaTaC formulation was 1833.0±211.2 pg/mL*h. This data shows that the bioavailability for the expandable device loaded capsule containing the C10 loaded formulation increased by about 6-fold compared to that of commercial tablets described in Example 6, while the bioavailability for the expandable device loaded capsule containing the NaTaC formulation increased by about 3.6-fold compared to that of commercial tablets described in Example 6. This difference is significant according to a 2-tailed t test and p value of 0.05.

### Example 9: In Vivo Imaging of the Expandable Device Transit Through GI Tract

In vivo beagle dog X-ray imaging experiments were performed at Shenzhen Advanced Medical Services Co., Ltd, China. Procedures involving the care and the use of animals in this study were reviewed and approved by the company Institutional Animal Care and Use Committee and performed according to the Chinese Animal Welfare Council guidelines and policies. X-ray imaging was performed in vivo on beagle dogs to monitor the opening as well as the transit through the GI tract of the 8 mm expandable device loaded in an enteric coated capsule as described in Example 3 containing no drug or excipient. To increase the contrast of the device under X-ray, short gold wires with a diameter of 500 μm and a length of 1.2 mm were embedded inside the elastomer materials forming a ring at the end of each device. After oral administration of the capsule, X-ray images were taken at the same time point as blood sampling, i.e., 0, 15, 30, 60, 90, 150, 240, and 360 min. X-ray images taken at the 30 min, 90 min, 150 min and 360 min time points are presented in FIGS. 8A-D. These data show that after 30 min, the device was fully expanded and migrating along the GI tract. These results show that the deployed expandable device could smoothly transit through the GI tract without disturbing the function of the intestine.

The scope of the claims should not be limited by the embodiments set forth in the examples, but should be given the broadest interpretation consistent with the description as a whole.

### REFERENCES

Dhalla, Arvinder K., Ziad Al-Shamsie, Simret Beraki, Anvesh Dasari, Leonard C. Fung, Laura Fusaro, Anusha Garapaty et al. “A robotic pill for oral delivery of biotherapeutics: safety, tolerability, and performance in healthy subjects.” Drug Delivery and Translational Research (2021): 1-12.
Dorkoosh, F. A., Verhoef, J. C., Verheijden, J. H. M., Rafiee-Tehrani M., Borchard, G., Junginger, H. E. Peroral absorption of octreotide in pigs formulated in delivery systems on the basis of superporous hydrogel polymers, Pharm. Res. 19 (2002) 1532-1536.
Granhall C, Donsmark M, Blicher™, Golor G, Søndergaard F L, Thomsen M, Bækdal TA. Safety and pharmacokinetics of single and multiple ascending doses of the novel oral human GLP-1 analogue, oral semaglutide, in healthy subjects and subjects with Type 2 Diabetes. Clin Pharmacokinet. 2019; 58:781-91.
Krauland A H, Guggi D, Bernkop-Schnürch A. (2004). Oral insulin delivery: the potential of thiolated chitosan-insulin tablets on non-diabetic rats. J Control Release, 95:547-555.
Melmed, R. N., A. A. A. EI-Aaser, and S. J. Holt. “Hypertrophy and hyperplasia of the neonatal rat exocrine pancreas induced by orally administered soybean trypsin inhibitor.” Biochimica et Biophysica Acta (BBA)-General Subjects 421, no. 2 (1976): 280-288.
Sam, M., Brayden, D. “Formulation strategies to improve the efficacy of intestinal permeation enhancers.” Adv. Drug Deliv. Rev (2021): 113925.

1. Swallowable, foldable and self-expandable oral drug delivery tubular device, the device:
comprising an outer peripheral surface;
having an expanded length of about 2 cm to about 6 cm;
having an expanded diameter of about 0.45 cm to about 6 cm; and
comprising at least one cavity enclosing a drug, the at least one cavity facing outward from the outer peripheral surface of the device.

2. The device of claim 1, wherein the tubular device comprises a single tubular mesh.

3. The device of claim 1, wherein the tubular device comprises foldable guiding members radially extending from a central tube or rod, each foldable guiding member comprising a guiding member outer peripheral surface, the combined guiding member outer peripheral surfaces of all foldable guiding members forming the device's outer peripheral surface.

4. The device of claim 1, wherein the drug is in a formulation further comprising at least one excipient.

5. The device of claim 4, wherein the at least one excipient comprises a permeation enhancer.

6. The device of claim 4, wherein the at least one excipient comprises a protease inhibitor.

7. The device of claim 1, wherein the drug has a molecular weight of about 1 kDa to about 50 kDa.

8. The device of claim 1, comprising drug cavities having a volume between 10 nl and 100 μL.

9. The device of claim 1, wherein the device material comprises or consists of an elastomer.

10. Enteric capsule enclosing the device defined in claim 1.

11. The capsule of claim 10, further comprising a filler.

12. The device of claim 1, for oral administration of the drug to a subject.

13. The device of capsule of claim 12, wherein the subject is a human and the device's expanded diameter is of about 1.1 cm to about 6 cm.

14. A method of using the device as defined in claim 1 enclosed in an enteric capsule, for the oral administration of the drug to a subject, the use comprising:
orally administering the capsule to the subject, whereby when the capsule releases the device in the small intestine, the device self-expands and positions itself coaxially in the intestine, and
whereby the drug migrates through the intestine epithelium and enters systemic circulation of the subject.

15. The use of claim 14, wherein the subject is a human.

16. A capsule enclosing the device defined in claim 1, for oral administration of the drug to a subject.
